(19) United States
(12) Patent Application Publication
(10) Pub. No.: US 2020/0069181 A1
(43) Pub. Date: Mar. 5, 2020

(54) I-Examination

(71) Applicant: Philip Jaques Sampson, Roselle, IL (US)

(72) Inventor: Philip Jaques Sampson, Roselle, IL (US)

(21) Appl. No.: 16/120,233

(22) Filed: Sep. 1, 2018

(51) Int. Cl. A61B 3/14; G06T 19/00; G06T 7/00; G02B 27/01; G16H 10/60

(52) U.S. Cl. CPC A61B 3/14; G06T 19/006; G06T 7/0012; G02B 2027/0178; G16H 10/60; G06T 2207/30041; G02B 27/0172

(57) ABSTRACT

Virtual reality (VR) technology uses the human eye as a medium to communicate with an individual. It allows for technological access to the wearer's eye which can be inspected to identify health issues to initiate preventative care. Potential health impact of early diagnosis includes reducing 600k deaths due to heart conditions. The foundation of I-Examination is improving the quality of health of those we hold dear, our family and friends.

VR is growing in popularity. People use it recreationally, viewing movies, conducting virtual tours, playing games, professionally for training or educational knowledge share. I-Examination, embedded within a VR, would have unlimited opportunities to examine the wearer's eyes. The application could run in the background as the individual engages in work or entertainment. It can also be directly applied during health assessments or routine eye examinations supporting an ophthalmologist to provide faster service effectively.

Current iris technology is already functional for biometrics supporting security, device log-on, and personal account management. Expanding this technology to the measure that I-Examination presents, VR scanning would present biometrics with opportunities to identify illnesses which are currently identifiable through eye exams. Patients who missed their annual eye examinations would still be presented with an opportunity for early diagnosis and a fighting chance to identify a health concern before it is dangerous or deadly.

### CROSS-REFERENCE TO RELATED APPLICATIONS

Virtual Reality glasses for watching movies or playing games.
Medical applications of patient portals connecting with wearable devices and physician's electronic health record systems.
Phones with cameras allowing for self-photography.
Biometric security using the iris of the eye.

### STATEMENT REGARDING FEDERALLY SPONSORED RESEARCH OR DEVELOPMENT

N/A

### THE NAMES OF THE PARTIES TO A JOINT RESEARCH AGREEMENT

N/A

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

N/A

### STATEMENT REGARDING PRIOR DISCLOSURES BY THE INVENTOR OR A JOINT INVENTOR

N/A

### BACKGROUND OF THE INVENTION

### (1) Field of the Invention

Healthcare field.

### (2) Description of Related Art including information disclosed under 37 CFR 1.97 and 1.98

### BRIEF SUMMARY OF THE INVENTION

Virtual reality technology uses the human eye as a medium to communicate with an individual. This allows for technological access to an individual's eye which can be inspected to identify health concerns. I-Examination will scan the individual's eyes as they use virtual reality for personal entertainment such as gaming or professional use such as training or be directly applied during health assessments. I-Examination will use current iris technology to securely access the individual's personal health information. I-Examination will present extensive opportunities to identify chronic diseases which are currently identifiable through eye exams such as diabetes, high cholesterol, and cancer.

### BRIEF DESCRIPTION OF THE SEVERAL VIEWS OF THE DRAWING(S)

A high-level depiction of I-Examination's architecture are included in the Diagram Document;

FIG. 0001—Diagram of an overview of the various components of I-Examination—Customer Interactive layer, Cloud layer and the Patient Interaction layer.

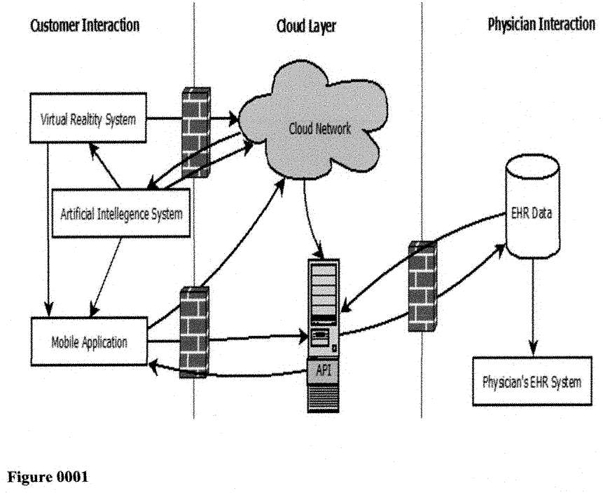

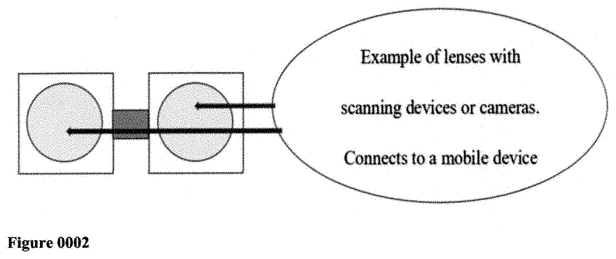

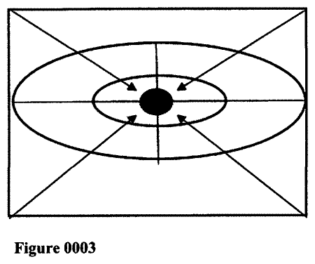

Description of the Cloud Layer component of I-Examination:—The VR glasses connect with the I-Examination cloud software using biometrics technology scanning the wearer's iris logging on securely. I-Examination will utilize artificial intelligence and a rule engine learning of anomalies for identifying illnesses with symptoms detectable in the eye. I-Examination's data connection with the mobile application and the physician's electronic health record (EHR) are managed through an API (application programming interface)

Description of the Physician Interaction Layer component of I-Examination:—I-Examination will directly integrate with the physician's EHR providing physicians health alerts of detected anomalies in the patient's eye and sharing images of the patient's eye. Physician's will be able to retrieve historic data from I-Examination to assess when the patient's health may have started deteriorating. For example, reviewing previous I-Examination findings to assess when the anomaly was first reported, or tracking I-Examination's updates to assess the patient's health progress for the impact of prescribed treatments.

FIG. 0002—Description of Step 1 the Customer Interface layer connecting the consumer through their mobile device with their wearable eye scanning device such as virtual reality glasses and their electronic health record system or application through and API (Application Programming Interface).

FIG. 0003. Description Step 2 of the Customer Interface layer connecting the consumer through their mobile device with their wearable eye scanning device such as virtual reality glasses and their electronic health record system or application through and API (Application Programming Interface).

Examples of identifiable symptoms of Diabetic Retinopathy include newly formed blood vessels and hemorrhages in the eye. For example, Identifiable symptoms of Hypertension include blood vessels in the eye exhibiting bends, kinks or tears.

Actions:
Alerts the wearer and their primary care physician.
Updates the Mobile app with results and supporting images.
Updates the wearer's electronic health record with findings, images and alerts.

### DETAILED DESCRIPTION OF THE INVENTION

I-Examination is a VR scanning interface requiring connectivity and interoperability between the web, mobile, VR, mobile phone and a physician's electronic health record system. The web and mobile application interface tracking the patient's health history provides information about their previous scans including dates, duration of the scan and the scanning results. The scan results may be in the form of scanned pictures or descriptions provided by I-Examination's scan result's diagnostic interpretations. The scan results transmit from the VR glasses to the web and mobile platforms daily and weekly connect with the patient's provider EHR system providing clinicians with pertinent information on the wearer's eye scan results. The VR scanner also connects with the wearer's cell phone cell phone to text an alert if an anomaly is detected within the wearer's eye. Integrating with AI (Artificial Intelligence) technology, the VR scanner continuously learns of new diseases identifiable through the eyes.

The mobile application interface contains embedded I-Examination functionality utilizing the mobile device's camera. The resultant exam quality, however, depends on the device's installed camera. The primary purpose of the device is the patient's accessing scan history, reports, analytical information and connectivity to other applications including patient portals and health tracking applications.

The VR device directly interacts with the patient's provider EHR through an API (Application Programming Interface). This interaction allows for the device to provide timely information to the patient's primary care physician of anomalies detected during an eye scan. The connection between the VR and the EHR must adhere to HIPAA (Health Insurance Portability and Accountability Act) regulations for the VR vendor and the EHR viewers as private health information will be exchanged.

Security is embedded within a trusted network relationship between the VR and the EHR. The patient's login to their VR occurs through biometric authentication via their eye as the VR can scan the wearer's iris for unique identification. In addition, the wearer would have security on their mobile device utilizing a fingerprint reader or a password. The double layered security should efficiently protect the patient's private health information from a security breach. The patient must approve text message alerts from the VR to their cell phone. This allows I-Examination to inform them that they are due for an eye scan or that an anomaly was detected during their last scan. Alerts of detected anomalies would be provided as a text message to patients, an email to patients, an email to their primary physicians' offices and display on their mobile patient applications and EHR portals. AI technology facilitates continuous machine learning for I-Examination to continue to improve as new methods for illness identification are encountered.

I-Examination engages the following resources: A VR expert, an AI expert, an API developer, an ophthalmologist, a physician, a project manager, a VR Programmer, a mobile programmer, an interface designer, a cloud developer, a database designer and developer, and patients to support usability and user acceptance testers. I-Examination requires the following capital resources: A camera, mobile devices such as an iPhone, an Android phone, an iPad, virtual reality glasses, EHR and a cloud server, Test environments for usability and user acceptance testing. I-Examination requires the following licensed software: Cloud technology and biometric security.

1. An eye examination technology, comprising:
a) An imaging device taking pictures or scanning the eye.
b) An imaging device on wearable devices like virtual reality glasses taking pictures or scanning the eye.
c) Means for inspecting images to identify health conditions including hypertension and diabetes.
d) A method for verifying personal identification wearing virtual reality glasses.
e) A method for exchanging information including images between a wearable eye imaging device and an individual's electronic health record system or application.
f) A method for applying artificial intelligence to assess health conditions based on images of the eye.
g) A method to connect with a mobile device to wearable eye imaging devices like virtual reality glasses.
h) A method to connect with a mobile device to an electronic health record system or application.
i) A method to use an application programming interface to connect devices such as a mobile device with an electronic health record system or virtual reality glasses with an electronic health record system.
